(19) United States
(12) Patent Application Publication
(10) Pub. No.: US 2008/0251079 A1
(43) Pub. Date: Oct. 16, 2008

(54) APPARATUS AND METHOD FOR PROVIDING POSITIVE AIRWAY PRESSURE

(75) Inventor: Joseph B. Richey, Chagrin Falls, OH (US)

Correspondence Address: CALFEE HALTER & GRISWOLD, LLP, 800 SUPERIOR AVENUE, SUITE 1400, CLEVELAND, OH, 44114, US

(73) Assignee: INVACARE CORPORATION, ELYRIA, OH (US)

(21) Appl. No.: 12/100,931

(22) Filed: Apr. 10, 2008

Related U.S. Application Data
(60) Provisional application No. 60/923,231, filed on Apr. 13, 2007.

(51) Int. Cl. A61M 16/00; A61M 16/06

(52) U.S. Cl. 128/204.26; 128/204.18; 128/205.25

(57) ABSTRACT

An apparatus for providing a breathing gas to a user may include: a headgear unit and a control unit. The headgear unit may include: a breathing interface, an adjustable structure to fit the headgear unit to the user's head with the breathing interface disposed in relation to the user's facial area, a blower motor assembly attached to the adjustable structure, and a plenum coupled to the breathing interface and the blower motor assembly to form a breathing gas flow path. The control unit controlling operation of the blower motor assembly based on a desired pressure for the breathing gas. Operation of the blower motor assembly provides the breathing gas to a user airway at an adjustable positive pressure via the breathing gas flow path. Additional embodiments of the apparatus and various embodiments of a related method are also provided herein.

### CROSS-REFERENCE TO RELATED APPLICATIONS

This application claims the benefit of U.S. Provisional Patent Application No. 60/923,231 (Attorney Docket Number 12873.05314), filed Apr. 13, 2007, the contents of which are fully incorporated herein by reference.

### BACKGROUND

Abnormal breathing may be treated by applying a breathing gas under positive pressure to a patient's airway via a positive airway pressure (PAP) device. This positive pressure may effectively “splint” the airway, thereby maintaining an open passage to the lungs. The pressure of the breathing gas delivered to the patient may be desired to be kept relatively constant at a desired or prescribed pressure during positive pressure therapy. This therapy technique is commonly referred to as constant positive airway pressure (CPAP). CPAP therapy may be provided using either open-loop or closed-loop control. CPAP therapy may be provided at a fixed target pressure using a control unit that controls breathing gas pressure based on the fixed target pressure. Alternatively, the CPAP therapy may also be controlled using a softened exhalation target pressure (SoftX™). SoftX™ is a trademark of Invacare Corporation. In SoftX™, the breathing gas is delivered at a relatively constant pressure, like CPAP, and during an initial portion of exhalation, the pressure set point is reduced, but then increases toward the constant pressure during the latter portion of exhalation, to help maintain the constant positive airway pressure.

In another type of positive pressure therapy, the pressure of the breathing gas delivered to the patient may be varied with the patient's breathing cycle or varied with the patient's effort such that the pressure during exhalation is less than the pressure during inhalation, This therapy technique may increase comfort to the patient during the therapy and is commonly referred to as bi-level positive airway pressure (BiPAP). In another type of positive pressure therapy, the pressure of the breathing gas delivered to the patient is varied in proportion to the flow generated by the patient. This therapy technique is commonly referred to as proportional positive airway pressure (PPAP).

Any of the various types of PAP devices may also incorporate ramping of the positive pressure from a lower pressure level to a higher desired or prescribed pressure level over an extended period (e.g., 10-15 minutes). This ramping process is intended to reduce the airway pressure while the patient is awake and for a period during which the patient may be expected to fall asleep. The positive airway pressure reaches the desired or prescribed level as the ramping period expires.

Likewise, any of the various types of PAP devices may also automatically adjust the level of pressure provided to the patient until reaching a minimum pressure or detecting an abnormal breathing condition, such as snoring or experiencing an apnea, hypopnea or upper airway resistance. If abnormal breathing is detected, the level of pressure may be increased until a maximum pressure is reached or the abnormal breathing condition is removed. This pressure support technique is sometimes referred to as auto-titration because the PAP device seeks to minimize the positive pressure provided to the patient to a level that is only as high as necessary to treat abnormal breathing conditions.

### SUMMARY

An exemplary headgear unit comprises a breathing interface in fluid communication with a blower motor assembly including a blower motor operatively connected to a blower for providing pressurized breathing gas to a user via the breathing interface, with the breathing interface and blower motor assembly being carried by the head of the user and with the blower motor and/or the blower being positioned proximate the crown of the user's head and/or the posterior of the user's head and/or the base of the user's skull, and/or the posterior of the user's neck. In the alternative, the blower motor and/or the blower may be positioned proximate the face of the user, or proximate the user's neck.

In one aspect, an exemplary apparatus for providing a breathing gas to a user is provided. In one exemplary embodiment, the apparatus may include: a headgear unit and a control unit in operative communication with the headgear unit. In this exemplary embodiment, the headgear unit includes: a breathing interface, an adjustable structure adapted to suitably fit the headgear unit to the user's head with the breathing interface disposed in operative relation to the user's facial area, a blower motor assembly releasably attached to the adjustable structure, and a plenum with a first end coupled to the breathing interface and an opposite end coupled to the blower motor assembly, the blower motor assembly, plenum, and breathing interface forming a breathing gas flow path. The plenum may be a conduit placing the blower in fluid connection with the breathing interface. The control unit may be used to selectively control operation of the blower motor assembly based at least in part on a desired pressure for the breathing gas. In this exemplary embodiment, operation of the blower motor assembly provides the breathing gas to at least one user airway at an adjustable positive pressure via the breathing gas flow path.

In another exemplary embodiment, the apparatus includes: a headgear unit and a control unit in operative communication with the headgear unit. The headgear unit may include: i) a breathing interface, ii) an adjustable structure adapted to suitably fit the headgear unit to the user's head with the breathing interface disposed in operative relation to the user's facial area, iii) a blower motor assembly releasably attached to the adjustable structure, and iv) a plenum with a first end coupled to the breathing interface and an opposite end coupled to the blower motor assembly. The blower motor assembly, plenum, and breathing interface forming a breathing gas flow path. The plenum may be a conduit placing the blower in fluid connection with the breathing interface. The control unit including: i) a first sensor in operative communication with the breathing gas flow path to sense a first characteristic associated with the breathing gas, ii) a closed loop control logic in operative communication with the first sensor and the blower motor assembly to selectively control operation of the blower motor assembly based at least in part on a desired pressure for the breathing gas and the first sensed characteristic, and iii) a desired pressure logic in operative communication with the first sensor and the closed loop control logic, the desired pressure logic detecting inhalation and exhalation periods of user breathing cycles based at least in part on the first sensed characteristic, wherein the desired pressure logic is adapted to adjust the desired pressure in relation to the detected inhalation and exhalation periods. In this embodiment, operation of the blower motor assembly provides the breathing gas to at least one user airway at an adjustable positive pressure via the breathing gas flow path.

In another exemplary aspect, an exemplary method for providing a breathing gas to a user is provided. In one embodiment the method may include: a) releasably attaching a blower motor assembly to an adjustable structure of a headgear unit, b) coupling a first end of a plenum to a breathing interface and an opposite end to the blower motor assembly to form a breathing gas flow path, c) adjusting the adjustable structure to suitably fit the headgear unit to the user's head with the breathing interface disposed in operative relation to the user's facial area, d) sensing a first characteristic associated with the breathing gas, and e) selectively controlling operation of the blower motor assembly in closed loop control fashion based at least in part on a desired pressure for the breathing gas and the first sensed characteristic to provide the breathing gas to at least one user airway at an adjustable positive pressure via the breathing gas flow path. The plenum may be a conduit placing the blower in fluid connection with the breathing interface.

### DRAWINGS

These and other features, aspects, and advantages of the present invention will become better understood with regard to the accompanying drawings, following description, and appended claims.

FIG. 1 provides a block diagram of an exemplary embodiment of a positive airway pressure (PAP) device;

FIG. 2 shows a perspective view of an exemplary embodiment of a headgear unit;

FIG. 3 provides a perspective view of another exemplary embodiment of a headgear unit;

FIG. 4 shows a front view of an exemplary embodiment of a control unit;

FIG. 5 provides a partial view of an exemplary embodiment of an interconnect assembly;

FIG. 6 shows a cross sectional view of the interconnect assembly of FIG. 5;

FIG. 7 provides a block diagram of another exemplary embodiment of a PAP device; and

FIG. 8 shows a flow chart of an exemplary embodiment of a process for providing a breathing gas to a user.

### DESCRIPTION

The following paragraphs include definitions of exemplary terms used within this disclosure. Except where noted otherwise, variants of all terms, including singular forms, plural forms, and other affixed forms, fall within each exemplary term meaning. Except where noted otherwise, capitalized and non-capitalized forms of all terms fall within each meaning.

“Circuit,” as used herein includes, but is not limited to, hardware, firmware, software or combinations of each to perform a function(s) or an action(s). For example, based on a desired feature or need, a circuit may include a software controlled microprocessor, discrete logic such as an application specific integrated circuit (ASIC), or another programmed logic device. A circuit may also be fully embodied as software. As used herein, “circuit” is considered synonymous with “logic.”

“Comprising,” “containing,” “having,” and “including,” as used herein, except where noted otherwise, are synonymous and open-ended. In other words, usage of any of these terms (or variants thereof does not exclude one or more additional elements or method steps from being added in combination with one or more delineated elements or method steps.

“Computer component,” as used herein includes, but is not limited to, a computer-related entity, either hardware, firmware, software, a combination thereof, or software in execution. For example, a computer component can be, but is not limited to being, a processor, an object, an executable, a process running on a processor a thread of execution, a program and a computer. By way of illustration, both an application running on a server and the server can be computer components. One or more computer components can reside within a process or thread of execution and a computer component can be localized on one computer or distributed between two or more computers.

“Computer communication,” as used herein includes, but is not limited to, a communication between two or more computer components and can be, for example, a network transfer, a file transfer, an applet transfer, an email, a hypertext transfer protocol (HTTP) message, a datagram, an object transfer, a binary large object (BLOB) transfer, and so on. A computer communication can occur across, for example, a wireless system (e.g., IEEE 802.11), an Ethernet system (e.g., IEEE 802.3), a token ring system (e.g., IEEE 802.5), a local area network (LAN), a wide area network (WAN), a point-to-point system, a circuit switching system, a packet switching system, and so on.

“Controller,” as used herein includes, but is not limited to, any circuit or device that coordinates and controls the operation of one or more input or output devices. For example, a controller can include a device having one or more processors, microprocessors, or central processing units (CPUs) capable of being programmed to perform input or output functions.

“Logic,” as used herein includes, but is not limited to, hardware, firmware, software or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software. As used herein, “logic” is considered synonymous with “circuit.”

“Measurement,” as used herein includes, but is not limited to, an extent, magnitude, size, capacity, amount, dimension, characteristic or quantity ascertained by measuring. Example measurements may be provided, but such examples are not intended to limit the scope of measurements that the systems and methods described herein can employ.

“Operable connection,” (or a connection by which entities are “operably connected” ), as used herein includes, but is not limited to, a connection in which signals, physical communication flow, or logical communication flow may be sent or received. Usually, an operable connection includes a physical interface, an electrical interface, or a data interface, but an operable connection may include differing combinations of these or other types of connections sufficient to allow operable control.

“Operative communication,” as used herein includes, but is not limited to, a communicative relationship between devices, logic, or circuits, including mechanical and pneumatic relationships. Direct and indirect electrical, electromagnetic, and optical connections are examples of connections that facilitate operative communications. Linkages, gears, chains, belts, push rods, cams, keys, attaching hardware, and other components contributing to mechanical relations between items are examples of components facilitating operative communications. Pneumatic devices and interconnecting pneumatic tubing may also contribute to operative communications. Two devices are in operative communication if an action from one causes an effect in the other, regardless of whether the action is modified by some other device. For example, two devices separated by one or more of the following: i) amplifiers, ii) filters, iii) transformers, iv) optical isolators, v) digital or analog buffers, vi) analog integrators, vii) other electronic circuitry, viii) fiber optic transceivers, ix) Bluetooth communications links, x) 802.11 communications links, xi) satellite communication links, and xii) other wireless communication links. As another example, an electromagnetic sensor is in operative communication with a signal if it receives electromagnetic radiation from the signal. As a final example, two devices not directly connected to each other, but both capable of interfacing with a third device, e.g., a central processing unit (CPU), are in operative communication.

“Or,” as used herein, except where noted otherwise, is inclusive, rather than exclusive. In other words, “or” is used to describe a list of alernative things in which one may choose one option or any combination of alternative options. For example, “A or B” means “A or B or both” and “A, B, or C” means “A, B, or C, in any combination.” If “or” is used to indicate an exclusive choice of alternatives or if there is any limitation on combinations of alternatives, the list of alternatives specifically indicates that choices are exclusive or that certain combinations are not included. For example, “A or B, but not both” is used to indicate use of an exclusive “or” condition. Similarly, “A, B, or C, but no combinations” and “A, B, or C, but not the combination of A, B, and C” are examples where certain combinations of alternatives are not included in the choices associated with the list.

“Processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read-only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

“Signal,” as used herein includes, but is not limited to, one or more electrical signals, including analog or digital signals, one or more computer instructions, a bit or bit stream, or the like.

“Software,” as used herein includes, but is not limited to, one or more computer readable or executable instructions that cause a computer or another electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system, or other types of executable instructions. It will be appreciated by one of ordinary skill in the art that the form of software is dependent on, for example, requirements of a desired application, the environment it runs on, or the desires of a designer/programmer or the like.

“Software component,” as used herein includes, but is not limited to, a collection of one or more computer readable or executable instructions that cause a computer or other electronic device to perform functions, actions or behave in a desired manner, The instructions may be embodied in various forms like routines, algorithms, modules, methods, threads, or programs. Software components may be implemented in a variety of executable or loadable forms including, but not limited to, a stand-alone program, a servelet, an applet, instructions stored in a memory, and the like. Software components can be embodied in a single computer component or can be distributed between computer components.

The following table includes long form definitions of exemplary acronyms used within this disclosure, Except where noted otherwise, variants of all acronyms, including singular forms, plural forms, and other affixed forms, fall within each exemplary acronym meaning. Except where noted otherwise, capitalized and non-capitalized forms of all acronyms fall within each meaning.

Acronym Long Form

ASIC Application specific integrated circuit

BLOB Binary large object

BiPAP Bi-level positive airway pressure

CPAP Constant positive airway pressure

CPU Central processing unit

DSP Digital signal processor

EPROM Erasable programmable read-only memory

HTTP Hypertext transfer protocol

LAN Local area network

LCD Liquid crystal display

Acronym Long Form

LED Light-emitting diode

PAP Positive airway pressure

PFL Persistent flow limitation

PPAP Proportional positive airway pressure

PROM Programmable read-only memory

PSG Polysomnogram

RAM Random access memory

ROM Read-only memory

RTV Room temperature vulcanizing

SoftX™ Softened exhalation target pressure (a brand of PAP)

WAN Wide area network

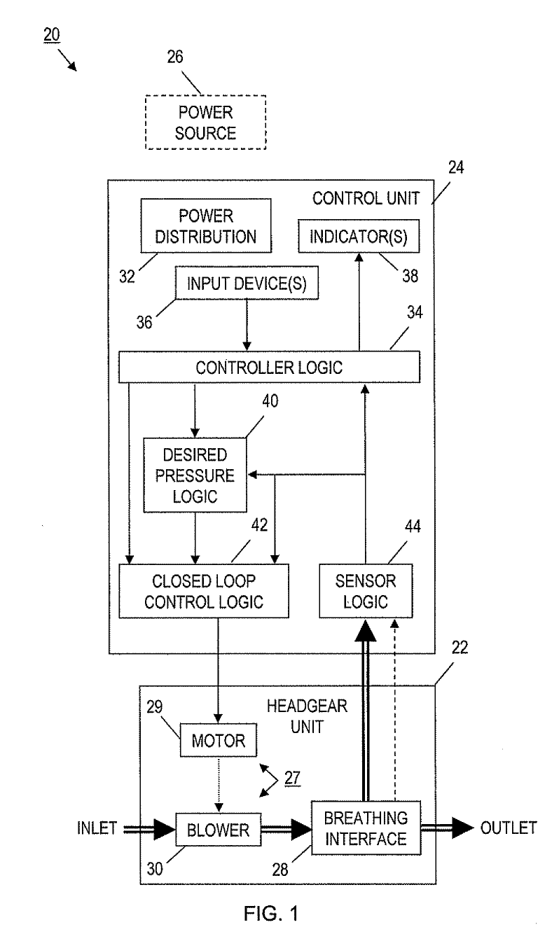

With reference to FIG. 1, an exemplary embodiment of a positive airway pressure (PAP) device 20 may include a headgear unit 22 and a control unit 24. The PAP device 20, for example, may include a CPAP device (i.e., standard CPAP, or CPAM with SoftX™, etc.), a BiPAP device, a PPAP device, an auto-titrating PAP device, a ventilator device, a gas therapy device, an oxygen therapy device, or another type of PAP device. Combinations of all these therapies are possible such as using any one therapy during inhalation and using any of the other therapies during exhalation, e.g., PPAP during inhalation and CPAP or no pressure splint during exhalation. The control unit 24 may be adapted to receive electrical power from any suitable power source 26, such as a utility power receptacle outlet, a utility power adapter, a battery pack, or another type of power storage device.

The headgear unit 22 may include a blower motor assembly 27 and a breathing interface 28. The blower motor assembly 27 may include a motor 29 and a blower 30. For example, a radial blower, such as model no. U64, manufactured by Micronel AG of Tagelswangen, Switzerland may be used as the blower motor assembly. The blower 30 may receive the breathing gas via an inlet. The blower 30 may be in fluid communication with the breathing interface 28 via, for example, a plenum or hose. The breathing interface 28 may include a nasal pillow or a nasal mask or a face mask or some other interface which may be fitted to a user. The motor 29 may be mechanically linked to the blower 30 such that operation of the motor 29 the blower 30 to pressurize the breathing gas which results in a flow of the breathing gas to the user via an outlet in the breathing interface 28. When the headgear unit 22 is properly fitted to the user, the breathing interface 28 is disposed in operative relation to the user's facial area such that the outlet provides the pressurized breathing gas to at least one user airway, such as the user's nasal or oral airway. The path from the inlet of the blower 30 to the outlet of the breathing interface 28 may be referred to as the breathing gas flow path.

The control unit may be very simple, such as a switch connecting the power source 26 to the blower motor 29 (in the case of an open-loop PAP or CPAP device). In the alternative, the control unit may have one or more sensors detecting some aspect of the fluid provided to the user (such as a pressure sensor detecting the pressure of air being provided to the user and/or a flow rate sensor sensing the rate of flow of air being provided to the user) and control logic to control some aspect of the fluid provided to the user, e.g., closed-loop CPAP, BiPAP, PPAP, etc. Exemplary control unit 24 is one of the latter types of control units with at least one sensor.

The exemplary control unit 24 may include a power distribution 32, a controller logic 34, one or more input devices 36, one or more indicators 38, a desired pressure logic 40, a closed loop control logic 42, and a sensor logic 44. The power distribution 32 distributes power to certain components of the PAP device 20. The distributed power may be switched, fused, filtered, or otherwise conditioned by the power distribution 32 for compatibility with desired operating modes and components to which power is distributed. When the power source 26 is utility power, the power distribution system may include an interface to a utility power receptacle outlet or a utility power adaptor. When the power source 26 is a power storage device (e.g., battery pack), the power distribution 32 may include an interface adapted to receive the power storage device. The control unit 24 may be equipped to receive power from both utility power and power storage device(s). If both utility power and power storage device(s) are received, the power distribution 32 may distribute utility power by default and charge the power storage device(s).

The controller logic 34 may be in communication with the one or more input devices 36, one or more indicators 38, desired pressure logic 40, closed loop control logic 42, and sensor logic 44. As shown, the controller logic 34 may be in operative communication with the motor 29 and breathing interface 28 of the headgear unit 22 via the closed loop control logic 42 and sensor logic 44, respectively. For example, the controller logic 34 may receive one or more signals from the one or more input devices 36 or sensor logic 44 in conjunction with controlling operation of the PAP device 20. The controller logic 34 may respond to such signals, for example, by starting operation of the blower motor assembly 27, controlling the speed of the motor 29 and blower 30 to control the pressure of the breathing gas in the breathing gas flow path, controlling the one or more indicators 38, or stopping operation of the blower motor assembly 27.

The one or more input devices 36, for example, may include operator switches to select between various operating modes (e.g., CPAP, CPAP with SoftX™, BiPAP, PPAP, etc.) or to select one or more desired or prescribed pressures (e.g., 10 cm H₂O, 2-40 cm H₂O, 10-28 cm H₂O, 15-20 cm H₂O). The controller logic 34, for example, may interactively control a display associated with the one or more indicators 38 to facilitate, for example, selection of a desired pressure using the one or more input devices 36. The sensor logic 44, for example, may be in fluid communication with the breathing gas flow path (e.g., breathing interface 28) and may include a pressure sensor and logic for detecting pressure in the breathing gas flow path. In another embodiment, the sensor logic 44 may be in electrical communication with the a pressure sensor in the breathing gas flow path (e.g., breathing interface 28). In other embodiments, one or more characteristics of the breathing gas related to pressure may be monitored and conditioned by the sensor logic 44 or the controller logic 34 to provide feedback to the closed loop control logic 42. For example, pressure, flow, and flow rate are examples of breathing gas characteristics related to pressure.

The controller logic 34 and desired pressure logic 40 may manipulate the desired and detected pressures. Ultimately, the closed loop control logic 42 may compare some representation of the detected pressure and the desired pressure and control the speed of the motor 29 to minimize the difference between the such pressures in closed loop control fashion. For example, when the standard CPAP operation mode is selected or intended, the closed loop control logic 42 may control the motor 29 to maintain a relatively constant positive pressure in the breathing gas flow path over at least one breathing cycle.

In another embodiment, the desired pressure, which is ultimately provided to the closed loop control logic 42, may gradually be increased by the controller logic 34 or desired pressure logic 40 from a reduced pressure level to the desired or prescribed pressure level over an extended period (e.g., 10-15 minutes). This is an implementation of ramping technology to delay application of the higher desired pressure until a time when the user may be expected to be sleeping.

In additional embodiments, the desired pressure provided to the closed loop control logic 42 may be adjusted to follow a breathing cycle pro-file based on selection of an operating mode for which the pressure is reduced for at least a portion of the exhalation period (e.g., CPAP with SoftX™ or BiPAP). In various embodiments, the resulting desired breathing cycle profile, sensors, and closed loop control scheme may be based at least partially on one or more characteristics that may be indicative of respiration (i.e., patient breathing) that are monitored by the sensor logic 44. For example, pressure, flow, flow rate, temperature, humidity, O₂, CO₂, motor Hall effect, motor voltage or current, motor speed, breathing gas valve position, and breathing gas vent position are examples of characteristics that may be indicative of respiration. Alternatively, the sensor logic 44 may monitor one or more patient physiological characteristic that may be indicative of respiration. For example, characteristics monitored during a polysomnogram (PSG) (i.e., a specific test used to diagnose sleep apnea) are examples of patient physiological characteristics that may be indicative of respiration.

Examples of various control schemes for PAP devices are disclosed in U.S. Pat. No. 6,990,980 to Richey II and U.S. patent application Ser. Nos. 10/601,720 and 11/157,089, both to Morris et al., all commonly assigned to Invacare Corporation, the contents of which are fully incorporated herein by reference. Any of the aspects of FIG. 1 described above may be automated, semi-automated, or manual and may be implemented through hardware, software, firmware, or combinations thereof.

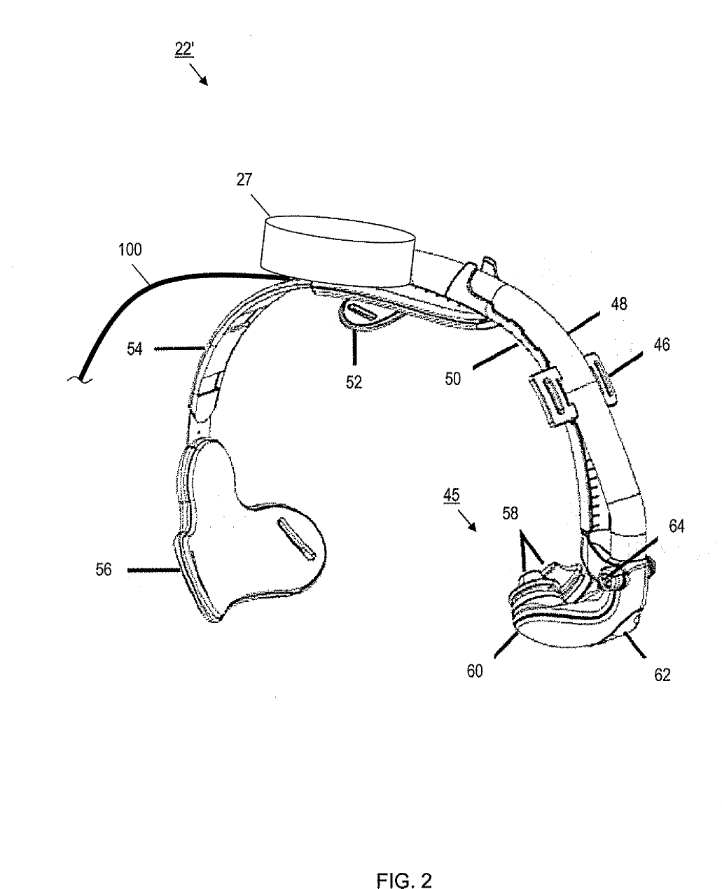

With reference to FIG. 2, an exemplary embodiment of a headgear unit 22′ may include a blower motor assembly 27, a nasal mask 45, a forward anchor 46, a plenum 48 (which may be a tube or other conduit), a guide 50, an upper anchor 52, an adjustable spine member 54, and a rear anchor 56. The nasal mask 45 may include a pair of nasal pillows 58, a shell 60, a vent 62, and an adjustable interconnect member 64 The rear anchor 56 and upper anchor 52 may be attached to the adjustable spine member 54. The adjustable spine member 54 may connect the upper anchor 52 and rear anchor 56 to the guide 50. The forward anchor 46 may be attached to the guide 50.

The forward anchor 46, guide 50, upper anchor 52, adjustable spine member 54, and rear anchor 56 may be substantially aligned along an axis of symmetry of the user's head. The adjustable spine member 54 may include a spring formed of spring steel. For example, the distance between the rear anchor 56 and the upper anchor 52 may be adjusted by extending or retracting extendable spines within a scabbard in the center portion of the adjustable spine member 54.

The adjustable spine member 54 may bias the rear anchor 56 against the user's occipital lobe, the upper anchor 52 against the user's crown, and the forward anchor 46 against the user's forehead so as to fit the headgear unit 22′ to the user's head. As such, the rear anchor 56, forward anchor 46, and upper anchor 52 may independently hold the headgear unit 22′ in place. In other embodiments, the eyelets in the rear anchor 56, forward anchor 46, and upper anchor 52 may be interconnected with various arrangements of straps or harnesses to provide additional support for holding the headgear unit 22′ in place. The straps or harnesses may include cloth or elastomeric material.

As shown, the blower motor assembly 27 may be releasably secured to the upper anchor 52. In other embodiments, the blower motor assembly 27 may be releasably or permanently secured to other components of the headgear unit 22′, such as the rear anchor 56, forward anchor 46, anywhere on the adjustable spine member 54, a strap between anchors (not shown), or a strap or rib associated with a harness (not shown). The plenum 46 is coupled to the nasal mask 45 and blower motor assembly 27 to form the breathing gas flow path. Placing the blower motor assembly 27 at or near the crown of the user's head permits the blower motor assembly 27 to be carried by the user without adding any additional moment on the user's head that would tend to cause the user's head to tilt while upright. Placing the blower motor assembly 27 at the back of the head or base of the user's skull permits the blower motor assembly 27 to provide a moment that tends to counteract the moment on the user's head created by the mass of the mask assembly or pillow assembly.

The guide 50 provides a location for securing the plenum 48 and nasal mask 45 to the headgear unit 22′. In another embodiment, the plenum 48 can be an integral part of the guide 50. The plenum 48 can have any suitable cross-sectional area and any suitable length. An exemplary range for the cross sectional area of the plenum 48 may be about 100 to 500 mm². An exemplary range for the length of the plenum 48 may be about 20 to 46 cm. In one exemplary embodiment, the plenum 48 may be less than about 25 centimeters long and may have a cross-sectional area of about 175 mm².

The nasal mask 45 may be formed by attaching the nasal pillows 58 and adjustable interconnect member 64 to the shell 60. The adjustable interconnect member 64 may couple the nasal mask 45 to the plenum 48 such that the nasal mask 45 can be adjusted along a vertical or horizontal axis to properly align the nasal pillows 58 to the user's nasal airway. During operation of the PAP device 20 (FIG. 1), carbon dioxide-rich gas exhaled by the user exits the breathing gas flow path through the vent 62. Generally, the vent 62 is sized so that positive pressure within the plenum 48 flushes the carbon dioxide-rich gas out the vent 62.

An exemplary embodiment of an interconnect assembly 100 is shown with the headgear unit 22′. The interconnect assembly 100 may facilitate fluid communication between the breathing gas flow path and the sensor logic 44 (FIG. 1) and may facilitate electrical communication between the blower motor assembly 27 and the closed loop control logic 42 (FIG. 1).

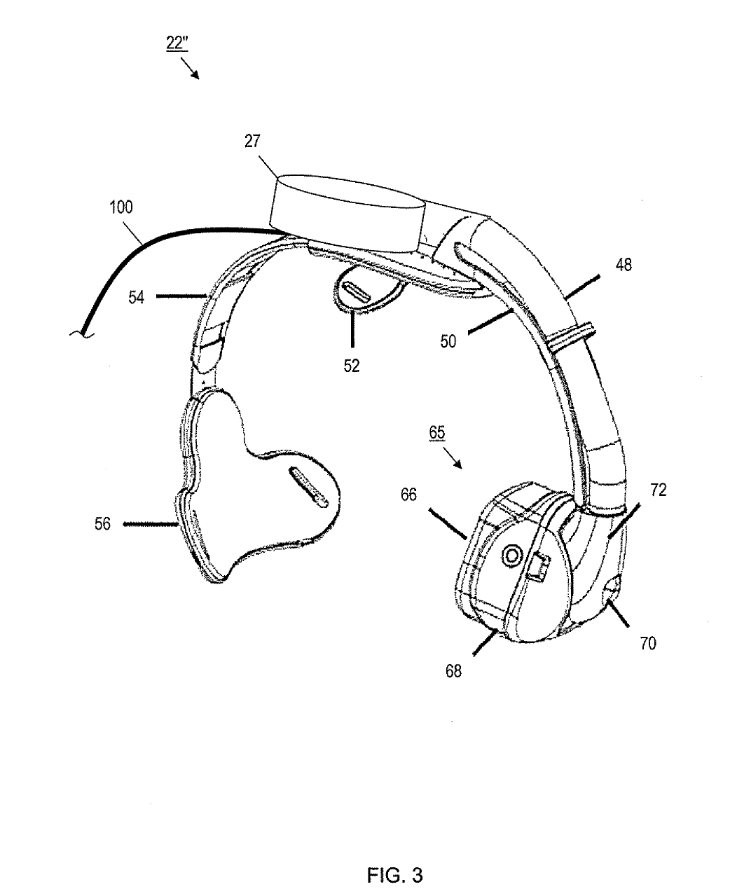

With reference to FIG. 3, another exemplary embodiment of a headgear unit 22″ may include a blower motor assembly 27, a plenum 48, a guide 50, an upper anchor 52, an adjustable spine member 54, a rear anchor 56, and a face mask 65. The face mask 65 may include a seal 66, a shell 68, a vent 70, and an adjustable interconnect member 72. The face mask 65 may be adapted to fit over user's nasal airway, oral airway, or both nasal and oral airways. The blower motor assembly 27, plenum 48, guide 50, upper anchor 52, adjustable spine member 54, rear anchor 56, and interconnect assembly 100 function in the same manner as described above for the headgear unit 22′ of FIG. 2.

As shown, the blower motor assembly 27 may be releasably secured to the upper anchor 52. In other embodiments, the blower motor assembly 27 may be releasably or permanently secured to other components of the headgear unit 22′, such as the rear anchor 56, a forward anchor, anywhere on the adjustable spine member 54, a strap between anchors (not shown), or a strap or rib associated with a harness (not shown). The plenum 46 is coupled to the nasal mask 45 and blower motor assembly 27 to form the breathing gas flow path. Placing the assembly 27 at or near the crown of the user's head permits the blower motor assembly 27 to be carried by the user without adding any additional moment on the user's head that would tend to cause the user's head to tilt while upright. Placing the blower motor assembly 27 at the back of the head or base of the user's skull permits the blower motor assembly 27 to provide a moment that tends to counteract the moment on the user's head created by the mass of the mask assembly or pillow assembly.

With continuing reference to FIG. 3, the face mask 65 is formed by attaching the seal 66 and adjustable interconnect member 72 to the shell 60. The adjustable interconnect member 72 couples the nasal mask 65 to the plenum 48 such that the nasal mask 65 can be adjusted along a horizontal or vertical axis to properly align the seal 66 with at least one of the user's airways and seat the seal in the corresponding facial area. During operation of the PAP device 20 (FIG. 1), carbon dioxide-rich gas exhaled by the user exits the breathing gas flow path through the vent 70. Generally, the vent 70 is sized so that positive pressure within the plenum 48 flushes the carbon dioxide-rich gas out the vent 70.

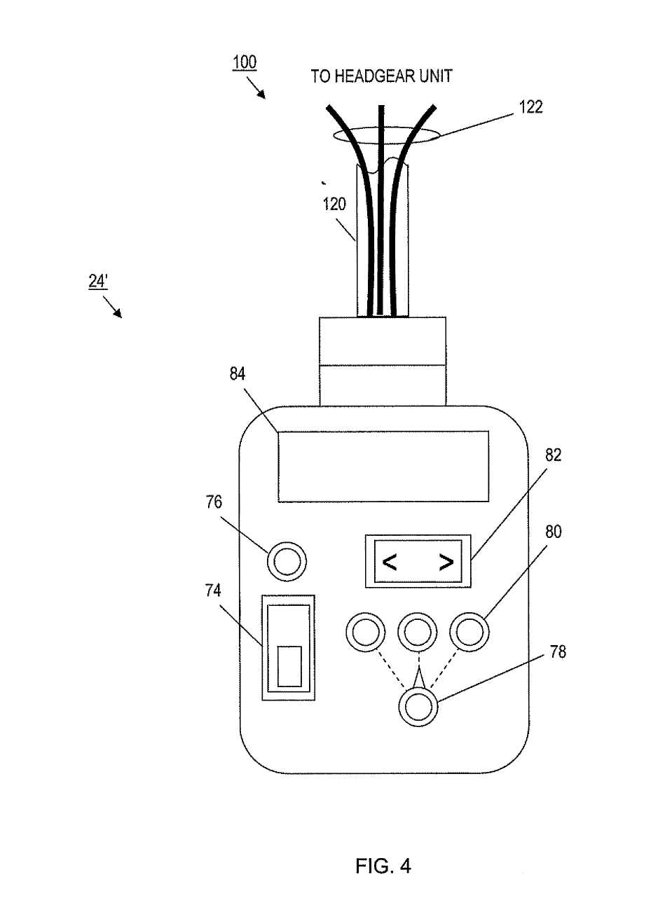

With reference to FIG. 4, an exemplary embodiment of a control unit 24′ may include a power switch 74, a power indicator 76, a mode switch 78, three mode indicators 80, an increase/decrease desired pressure switch 82, and a display 84. The power switch 74 and power indicator 76 may be part of an exemplary power distribution 32 (FIG. 1). The power switch 74 may include any suitable switch which is operated to connect and disconnect power between power distribution 32 (FIG. 1) and other components of the PAP device 20 (FIG. 1). The power switch 74, for example, may include a slide switch, toggle switch, pushbutton switch, rotary switch, or any other suitable switch, The power indicator 76 may include any suitable indicator light that is illuminated when power is connected from power distribution 32 (FIG. 1) to other components of the PAP device 20 (FIG. 1) and extinguished when power is disconnected. For example, the power indicator 76 may include a light-emitting diode (LED), an incandescent lamp, or any other suitable indicator.

The mode switch 82 and increase/decrease desired pressure switch 82 are examples of one or more input devices 36 (FIG. 1). Similarly, the three mode indicators 80 and display 84 are examples of one or more indicators 38 (FIG. 1). The mode switch may be a combination rotary pushbutton switch to select between alternate modes of operation for the PAP device 20 (FIG. 1), such as setup, standard CPAP, CPAP with SoftX™, and BiPAP. The rotary switch portion, for example, may include a 3-position rotary switch or any suitable multi-position switch with at least one position for each available operating mode. In other embodiments, operating modes may be selected by combinations of one or more pushbutton or toggle switches. The pushbutton switch portion of the mode switch may include a center pushbutton that activates a setup mode so that a desired pressure associated with the operating mode selected by the rotary switch portion may be input using, for example, the increase/decrease desired pressure switch 82. Setup mode may be a secured mode, for example, where the pushbutton switch is key-operated. In other embodiments, any suitable form of hardware or software security that limits setup mode access to authorized users may be implemented. In additional embodiments, various schemes of input devices and software controls may be implemented to select operating modes.

During setup mode, the increase/decrease desired pressure switch 82 may be activated to increase or decrease the desired or prescribed pressure associated with a selected normal operating mode (e.g., CPAP). For example, the desired pressure may be updated on the display 84 as the increase/decrease desired pressure switch 82 is activated. The increase/decrease desired pressure switch 82, for example, may include a 2-position momentary return-to-center switch or any suitable switch or combination of switches. The display 84 may include a liquid crystal display (LCD), numeric display, graphic display, or any suitable display. During the normal operating modes, the display 84 may indicate either the desired pressure, detected pressure, or both pressures. In one embodiment, alternating activations of the increase and decrease functions of the desired pressure switch 82 during normal operating modes may toggle the display 84 between showing the desired pressure and the detected pressure.

An exemplary embodiment of an interconnect assembly 100 is shown with the control unit 24′. As shown, the interconnect assembly 100 may include a conduit 120 and a plurality of electrical conductors 122. The conduit 120 may facilitate fluid communication between the sensor logic 44 (FIG. 1) and the breathing gas flow path and the plurality of electrical conductors 122 may facilitate electrical communication between the closed loop control logic 42 (FIG. 1) and the motor 29 (FIG. 1). Any of the aspects of FIG. 4 or related embodiments described above may be automated, semi-automated, or manual and may be implemented through hardware, software, firmware, or combinations thereof.

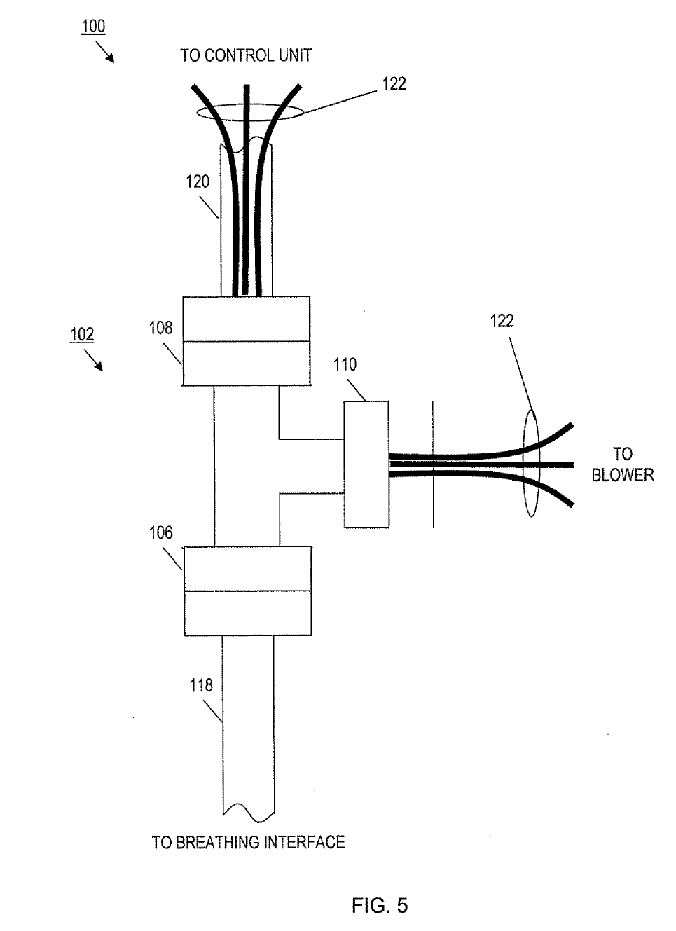

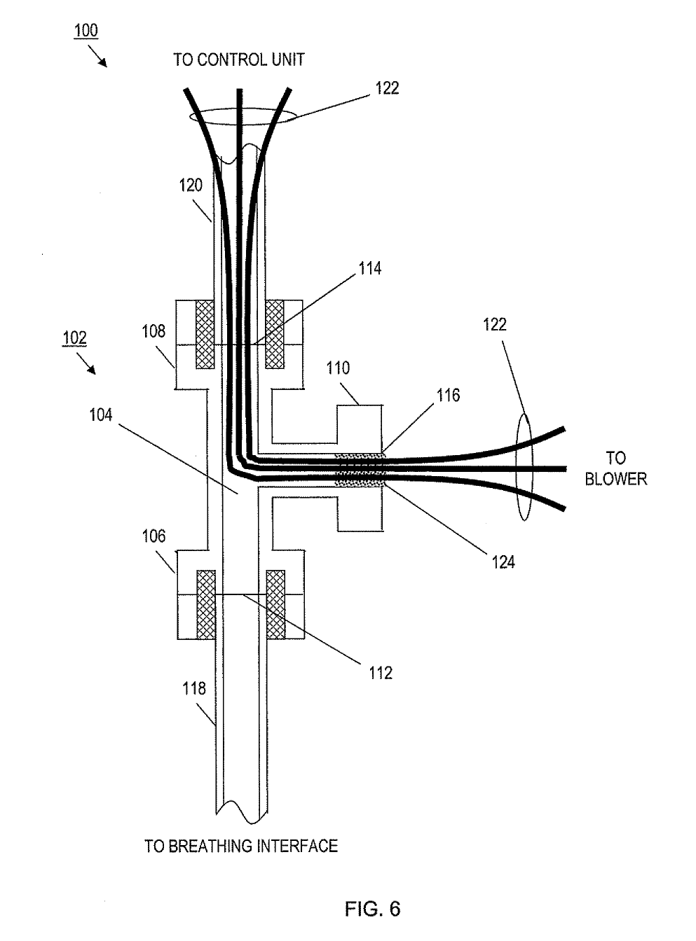

With reference to FIGS. 5 and 6, an exemplary embodiment of an interconnect assembly 100 may include a fitting 102 having an interior cavity 104 and first, second, and third ports 106, 108, 110 with corresponding through apertures 112, 114, 116 to the interior cavity 104. The interconnect assembly 100 may also include first and second conduits 118, 120. The first conduit 118 may include a first end coupled to the breathing gas flow path in the headgear unit 22 (FIG. 1) and an opposite end coupled to the first port 106. The second conduit 120 may include a first end coupled to the second port 108 and an opposite end operatively communicated to the sensor logic 44 (FIG. 1) in the control unit 24 (FIG. 1). The interconnect assembly 100 may also include a plurality of electrical conductors 122 i) operatively communicated to the closed loop control logic 42 (FIG. 1) in the control unit 24 (FIG. 1), ii) routed through the second conduit 122 to the fitting 102, iii) routed through the aperture 114 of the second port 108, interior cavity 104 of the fitting 102, and aperture 116 of the third port 110, and iv) operatively communicated to the blower motor assembly 27 (FIG. 1) in the headgear unit 22 (FIG. 1).

The aperture 116 in the third port 110 may be suitably sealed by the plurality of electrical conductors 122 in combination with a fill material 124 such that the first conduit 118, fitting 104, and second conduit 122 form a fluid path from the breathing gas flow path to the control unit 24 (FIG. 1). The fill material 124, for example, may include a room temperature vulcanizing (RTV) material or the like. In another embodiment, at least the portion of the electrical conductors 122 retained within the fitting 102 may be suitably protected from chaffing using shrink wrap, strain relief techniques, and similar practices to bulk up insulation of the wire and limit movement of the conductors within the fitting 102 in the final manufactured assembly.

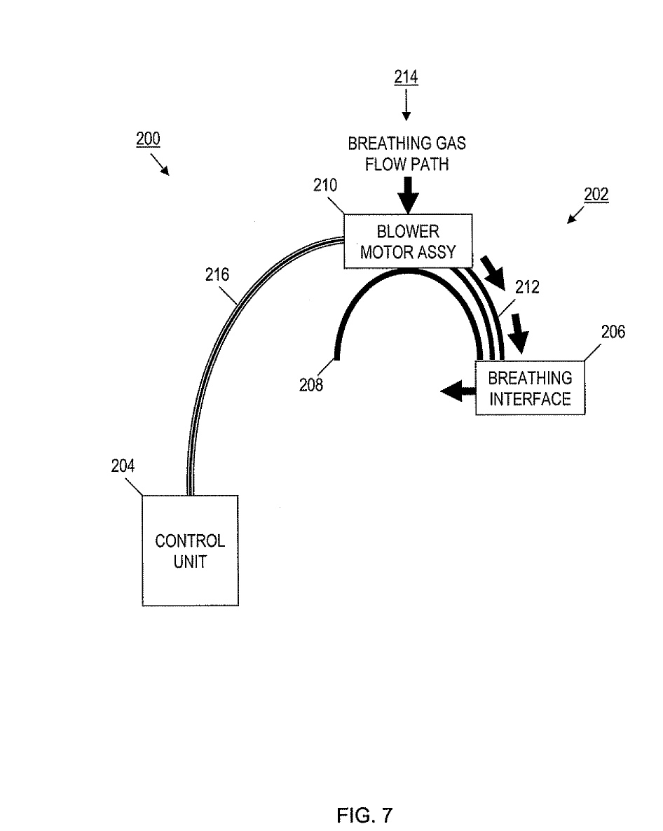

With reference to FIG. 7, another exemplary embodiment of a PAP device 200 for providing a breathing gas to a user may include a headgear unit 202 and a control unit 204 in operative communication with the headgear unit 202. In this embodiment, the headgear unit 202 may include a breathing interface 206 and an adjustable structure 208 adapted to suitably fit the headgear unit 202 to the user's head with the breathing interface 206 disposed in operative relation to the user's facial area. The headgear unit 202 may also include a blower motor assembly 210 releasably attached to the adjustable structure 208 and a plenum 212 with a first end coupled to the breathing interface 206 and an opposite end coupled to the blower motor assembly 210. The blower motor assembly 210, plenum 212, and breathing interface 206 form a breathing gas flow path 214. The blower motor assembly 210 may be placed in any of many locations in or proximate to the adjustable structure 208 such as at the crown of the head, anywhere at the posterior of the proximate the adjustable structure 208, such as proximate the crown of the head, anywhere on the posterior of the head, the base of the skull, back of the neck, etc. In the alternative, the blower motor assembly 210 may be placed proximate the face or the neck of the user. In this exemplary embodiment, the control unit 204 may selectively control operation of the blower motor assembly 210 based at least in part on a desired pressure for the breathing gas. Operation of the blower motor assembly 210 may provide the breathing gas to at least one user airway at an adjustable positive pressure via the breathing gas flow path 214.

In another exemplary embodiment, the PAP device 200 may include a first sensor in operative communication with the breathing gas flow path 214 to sense a first characteristic associated with the breathing gas. In this embodiment, the control unit 204 may include a closed loop control logic 42 (FIG. 1) in operative communication with the first sensor and the blower motor assembly 210 to selectively control operation of the blower motor assembly 210 based at least in part on the desired pressure and the first sensed characteristic. In a still another embodiment, the closed loop control logic 42 (FIG. 1) may selectively control the blower motor assembly 210 to maintain a relatively constant positive pressure in the breathing gas flow path 210 over a span of at least one user breathing cycle. In various embodiments, the first sensor may include a pressure sensor, a flow sensor, a flow rate sensor, a temperature sensor, a humidity sensor, an O₂ sensor, a CO₂ sensor, a motor Hall effect sensor, a motor voltage or current sensor, a motor speed sensor, a breathing gas valve position sensor, or a breathing gas vent position sensor. Alternatively, the first sensor may sense one or more patient physiological characteristic that may be indicative of respiration. For example, characteristics monitored during a PSG are examples of patient physiological characteristics that may be indicative of respiration.

In yet another exemplary embodiment, control unit may also include a desired pressure logic 40 (FIG. 1) in operative communication with the first sensor and the closed loop control logic 42 (FIG. 1). The desired pressure logic 40 (FIG. 1) may detect inhalation and exhalation periods of user breathing cycles based at least in part on the first sensed characteristic. The desired pressure logic 40 (FIG. 1) may also be adapted to adjust the desired pressure in relation to the detected inhalation and exhalation periods. In still yet another embodiment, the desired pressure logic 40 (FIG. 1) may adjust the desired pressure over a span of at least two consecutive user breathing cycles as a function of the detected inhalation and exhalation periods such that the desired pressure is set to a normal level during detected inhalation periods and set to a reduced level during detected exhalation periods. In another embodiment, the desired pressure logic 40 (FIG. 1) may adjust the desired pressure over a span of at least two consecutive user breathing cycles as a function of the detected inhalation and exhalation periods such that the desired pressure is set to a normal level during detected inhalation periods, set to a reduced level during an initial portion of detected exhalation periods, and gradually increased from the reduced level to the normal level during a remaining portion of detected exhalation periods. In yet another exemplary embodiment, the desired pressure logic 40 (FIG. 1) may detect at least one type of abnormal user breathing based at least in part on the first sensed characteristic and may adjust the desired pressure over time to decrease the desired pressure until either a minimum pressure is reached or abnormal user breathing is detected.

In still another exemplary embodiment, the PAP device 200 may also include a second sensor in operative communication with the breathing gas flow path 216 to sense a second characteristic associated with the breathing gas. In this embodiment, the control unit 204 may also include a desired pressure logic 40 (FIG. 1) in operative communication with the second sensor and the closed loop control logic 42 (FIG. 1). The desired pressure logic 40 (FIG. 1) may detect inhalation and exhalation periods of user breathing cycles based at least in part on the second sensed characteristic. The desired pressure logic 40 (FIG. 1) may also be adapted to adjust the desired pressure in relation to the detected inhalation and exhalation periods. Additionally, the desired pressure logic 40 (FIG. 1) may detect at least one type of abnormal user breathing based at least in part on the second sensed characteristic and may adjust the desired pressure over time to decrease the desired pressure until either a minimum pressure is reached or abnormal user breathing is detected. In various embodiments, the second sensor may include a pressure sensor, a flow sensor, a flow rate sensor, a temperature sensor, a humidity sensor, an O₂ sensor, a CO₂ sensor, a motor Hall effect sensor, a motor voltage or current sensor, a motor speed sensor, a breathing gas valve position sensor, or a breathing gas vent position sensor. Alternatively, the first sensor may sense one or more patient physiological characteristic that may be indicative of respiration. For example, characteristics monitored during a PSG are examples of patient physiological characteristics that may be indicative of respiration.

In still yet another embodiment, where the first sensor is disposed within the control unit 204, such as in a sensor logic 44 (FIG. 1), the PAP device 200 may also include an interconnect assembly 216 facilitating fluid communication between the breathing gas flow path 214 and the first sensor and facilitating electrical communication between the closed loop control logic 42 (FIG. 1) and the blower motor assembly 210. In another embodiment, the breathing interface 206 may include a nasal mask 45 (FIG. 2) adapted for positioning in relation to the user's nose to provide the breathing gas to the user at a nasal airway. In yet another embodiment, the breathing interface 206 may include a face mask 65 (FIG. 3) adapted for positioning in relation to at least one of the user's nose and mouth to provide the breathing gas to the user via at least one of a nasal airway and an oral airway.

In still another exemplary embodiment, the blower motor assembly 210 may include a brushless DC motor adapted to rotate at various predetermined speeds in response to adjustable alternating signals from the control unit 204. In still yet another embodiment, the blower motor assembly 210 is releasably attached to the adjustable structure 208 at a location proximate a crown of the user's head. Any of the aspects of FIG. 7 or related embodiments described above may be automated, semi-automated, or manual and may be implemented through hardware, software, firmware, or combinations thereof.

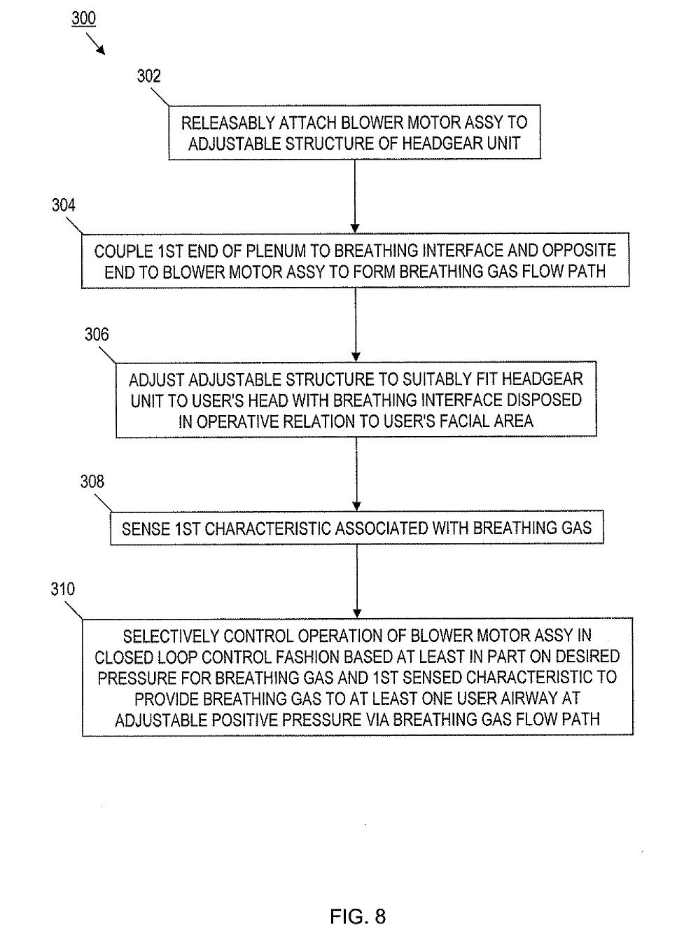

With reference to FIG. 8, an exemplary embodiment of a process 300 for providing a breathing gas to a user begins at 302 where a blower motor assembly may be releasably attached to an adjustable structure of a headgear unit. At 304, a first end of a plenum may be coupled to a breathing interface and an opposite end may be coupled to the blower motor assembly to form a breathing gas flow path. Next, the adjustable structure may be adjusted to suitably fit the headgear unit to the user's head with the breathing interface disposed in operative relation to the user's facial area (306). At 308, a first characteristic associated with the breathing gas may be sensed. In various embodiments, the first sensed characteristic may include pressure, flow, flow rate, temperature, humidity, O₂, CO₂, motor Hall effect, motor voltage or current, motor speed, breathing gas valve position, or breathing gas vent position. Alternatively, the first sensed characteristic may include one or more patient physiological characteristics that may be indicative of respiration. For example, characteristics monitored during a PSG are examples of patient physiological characteristics that may be indicative of respiration. Next, operation of the blower motor assembly may be selectively controlled in closed loop control fashion based at least in part on a desired pressure for the breathing gas and the first sensed characteristic to provide the breathing gas to at least one user airway at an adjustable positive pressure via the breathing gas flow path (310).

In another exemplary embodiment, the controlling in 310 may selectively control the blower motor assembly to maintain a relatively constant positive pressure in the breathing gas flow path over a span of at least one user breathing cycle. In yet another embodiment, the process 300 may also include detecting inhalation and exhalation periods of user breathing cycles based at least in part on the first sensed characteristic and adjusting the desired pressure over a span of at least two consecutive user breathing cycles as a function of the detected inhalation and exhalation periods. In still another exemplary embodiment, the process 300 may include detecting at least one type of abnormal user breathing based at least in part on the first sensed characteristic and adjusting the desired pressure over time to decrease the desired pressure until either a minimum pressure is reached or abnormal user breathing is detected.

In still yet another exemplary embodiment, the process 300 may include sensing a second characteristic associated with the breathing gas. In various embodiments, the second sensed characteristic may include pressure, flow, flow rate, temperature, humidity, O₂, CO₂, motor Hall effect, motor voltage or current, motor speed, breathing gas valve position, breathing gas vent position. Alternatively, the first sensed characteristic may include one or more patient physiological characteristics that may be indicative of respiration. For example, characteristics monitored during a PSG are examples of patient physiological characteristics that may be indicative of respiration. In this embodiment, inhalation and exhalation periods of user breathing cycles may be detected based at least in part on the second sensed characteristic. Additionally, the desired pressure may be adjusted in relation to the detected inhalation and exhalation periods. At least one type of abnormal user breathing may also be detected based at least in part on the second sensed characteristic. The desired pressure may also be adjusted over time to decrease the desired pressure until either a minimum pressure is reached or abnormal user breathing is detected. Any of the aspects of FIG. 8 or related embodiments described above may be automated, semi-automated, or manual and may be implemented through hardware, software, firmware, or combinations thereof.

While the invention is described herein in conjunction with one or more exemplary embodiments, it is evident that many alternatives, modifications, and variations will be apparent to those skilled in the art. For example, although the various embodiments are discussed with respect to the motor/blower assembly being positioned in one location or another, these parts may be located separately in any of the various locations and operatively connected. As another example, in any of the various embodiments, humidification of breathing gas may be provided with an optional humidifier, which may include a mister or other misting device at some location between the blower inlet and the user interface, e.g., in tubing or another conduit between the blower and the user interface. Accordingly, exemplary embodiments in the preceding description are intended to be illustrative, rather than limiting, of the spirit and scope of the invention. More specifically, it is intended that the invention embrace all alternatives, modifications, and variations of the exemplary embodiments described herein that fall within the spirit and scope of the appended claims or the equivalents thereof. Any element in a claim that does not explicitly state “means for” performing a specified function, or “step for” performing a specific function, is not to be interpreted as a “means” or “step” clause as specified in 35 U.S.C. §112, ¶6. In particular, the use of “step of” in the claims herein is not intended to invoke the provisions of 35 U.S.C. §112, ¶6.

1. An apparatus for providing a breathing gas to a user, including:
a headgear unit, including:
a breathing interface;
an adjustable structure adapted to suitably fit the headgear unit to the user's head with the breathing interface disposed in operative relation to the user's facial area;
a blower motor assembly releasably attached to the adjustable structure positioning the assembly at the crown of or at a posterior portion of the user's head or neck; and
a plenum with a first end coupled to the breathing interface and an opposite end coupled to the blower motor assembly, the blower motor assembly, plenum, and breathing interface forming a breathing gas flow path; and
a control unit in operative communication with the headgear unit to selectively control operation of the blower motor assembly based at least in part on a desired pressure for the breathing gas;
wherein operation of the blower motor assembly provides the breathing gas to at least one user airway at an adjustable positive pressure via the breathing gas flow path.

2. The apparatus of claim 1, further including:
a first sensor in operative communication with the breathing gas flow path to sense a first characteristic associated with the breathing gas; and
the control unit including:
a closed loop control logic in operative communication with the first sensor and the blower motor assembly to selectively control operation of the blower motor assembly based at least in part on the desired pressure and the first sensed characteristic.

3. The apparatus of claim 2 wherein the closed loop control logic selectively controls the blower motor assembly to maintain a relatively constant positive pressure in the breathing gas flow path over a span of at least one user breathing cycle.

4. The apparatus of claim 2, the control unit further including:
a desired pressure logic in operative communication with the first sensor and the closed loop control logic, the desired pressure logic detecting inhalation and exhalation periods of user breathing cycles based at least in part on the first sensed characteristic, wherein the desired pressure logic is adapted to adjust the desired pressure in relation to the detected inhalation and exhalation periods.

5. The apparatus of claim 4 wherein the desired pressure logic adjusts the desired pressure over a span of at least two consecutive user breathing cycles as a function of the detected inhalation and exhalation periods such that the desired pressure is set to a normal level during detected inhalation periods and set to a reduced level during detected exhalation periods.

6. The apparatus of claim 4 wherein the desired pressure logic adjusts the desired pressure over a span of at least two consecutive user breathing cycles as a function of the detected inhalation and exhalation periods such that the desired pressure is set to a normal level during detected inhalation periods, set to a reduced level during an initial portion of detected exhalation periods, and gradually increased from the reduced level to the normal level during a remaining portion of detected exhalation periods.

7. The apparatus of claim 4 wherein the desired pressure logic detects at least one type of abnormal user breathing based at least in part on the first sensed characteristic and adjusts the desired pressure over time to decrease the desired pressure until either a minimum pressure is reached or abnormal user breathing is detected.

8. The apparatus of claim 2, further including:
a second sensor in operative communication with the breathing gas flow path to sense a second characteristic associated with the breathing gas; and
the control unit further including:
a desired pressure logic in operative communication with the second sensor and the closed loop control logic, the desired pressure logic detecting inhalation and exhalation periods of user breathing cycles based at least in part on the second sensed characteristic, wherein the desired pressure logic is adapted to adjust the desired pressure in relation to the detected inhalation and exhalation periods;
wherein the desired pressure logic detects at least one type of abnormal user breathing based at least in part on the second sensed characteristic and adjusts the desired pressure over time to decrease the desired pressure until either a minimum pressure is reached or abnormal user breathing is detected.

9. The apparatus of claim 2 wherein the first sensor is disposed within the control unit, the apparatus further including;
an interconnect assembly facilitating fluid communication between the breathing gas flow path and the first sensor and facilitating electrical communication between the closed loop control logic and the blower motor assembly.

10. The apparatus of claim 9, the interconnect assembly including:
a fitting having an interior cavity and first, second, and third ports with corresponding through apertures to the interior cavity;
a first conduit having a first end coupled to the breathing gas flow path and an opposite end coupled to the first port;
a second conduit having a first end coupled to the second port and an opposite end operatively communicated to the first sensor; and
a plurality of electrical conductors i) operatively communicated to the closed loop control logic, ii) routed through the second conduit to the fitting, iii) routed through the aperture of the second port, interior cavity of the fitting, and aperture of the third port, and iv) operatively communicated to the blower motor assembly;
wherein the aperture in the third port is suitably sealed by the plurality of electrical conductors in combination with a fill material such that the first conduit, fitting, and second conduit form a fluid path from the breathing gas flow path to the control unit.

11. The apparatus of claim 1, the breathing interface including:
a nasal mask adapted for positioning in relation to the user's nose to provide the breathing gas to the user at a nasal airway.

12. The apparatus of claim 1, the breathing interface including:
a face mask adapted for positioning in relation to at least one of the user's nose and mouth to provide the breathing gas to the user via at least one of a nasal airway and an oral airway.

13. The apparatus of claim 1, the blower motor assembly including:
a brushless DC motor adapted to rotate at various predetermined speeds in response to adjustable alternating signals from the control unit.

14. The apparatus of claim 1 wherein the blower motor assembly is releasably attached to the adjustable structure at a location proximate a crown of the user's head.

15. The apparatus of claim 1 wherein the blower motor assembly is releasably attached to the adjustable structure at a location proximate the rear of the user's head.

16. The apparatus of claim 1 wherein the blower motor assembly is releasably attached to the adjustable structure at a location proximate the base of the user's skull.

17. The apparatus of claim 1, further including:
a first sensor in operative communication with the breathing gas flow path to sense a first characteristic associated with the breathing gas, wherein the first sensor is disposed within the control unit;
the control unit including:
a closed loop control logic in operative communication with the first sensor and the blower motor assembly to selectively control operation of the blower motor assembly based at least in part on the desired pressure and the first sensed characteristic;
the apparatus farther including:
a desired pressure logic in operative communication with the first sensor and the closed loop control logic, the desired pressure logic detecting inhalation and exhalation periods of user breathing cycles based at least in part on the first sensed characteristic, wherein the desired pressure logic is adapted to adjust the desired pressure in relation to the detected inhalation and exhalation periods, wherein the desired pressure logic detects at least one type of abnormal user breathing based at least in part on the first sensed characteristic and adjusts the desired pressure over time to decrease the desired pressure until either a minimum pressure is reached or abnormal user breathing is detected; and
an interconnect assembly facilitating fluid communication between the breathing gas flow path and the first sensor and facilitating electrical communication between the closed loop control logic and the blower motor assembly, the interconnect assembly including:
a fitting having an interior cavity and first, second, and third ports with corresponding through apertures to the interior cavity;
a first conduit having a first end coupled to the breathing gas flow path and an opposite end coupled to the first port;
a second conduit having a first end coupled to the second port and an opposite end operatively communicated to the first sensor; and
a plurality of electrical conductors i) operatively communicated to the closed loop control logic, ii) routed through the second conduit to the fitting, iii) routed through the aperture of the second port, interior cavity of the fitting, and aperture of the third port, and iv) operatively communicated to the blower motor assembly;
wherein the aperture in the third port is suitably sealed by the plurality of electrical conductors in combination with a fill material such that the first conduit, fitting, and second conduit form a fluid path from the breathing gas flow path to the control unit; and
the blower motor assembly including:
a brushless DC motor adapted to rotate at various predetermined speeds in response to adjustable alternating signals from the control unit.

18. A method for providing a breathing gas to a user, including:
a) releasably attaching a blower motor assembly to an adjustable structure of a headgear unit positioning the assembly at the crown of or at a posterior portion of the user's head or neck;
b) coupling a first end of a plenum to a breathing interface and an opposite end to the blower motor assembly to form a breathing gas flow path;
c) adjusting the adjustable structure to suitably fit the headgear unit to the user's head with the breathing interface disposed in operative relation to the user's facial area;
d) sensing a first characteristic associated with the breathing gas; and
e) selectively controlling operation of the blower motor assembly in closed loop control fashion based at least in part on a desired pressure for the breathing gas and the first sensed characteristic to provide the breathing gas to at least one user airway at an adjustable positive pressure via the breathing gas flow path.

19. The method of claim 18 wherein the controlling in e) selectively controls the blower motor assembly to maintain a relatively constant positive pressure in the breathing gas flow path over a span of at least one user breathing cycle.

20. The method of claim 18, further including:
f) detecting inhalation and exhalation periods of user breathing cycles based at least in part on the first sensed characteristic; and
g) adjusting the desired pressure over a span of at least two consecutive user breathing cycles as a function of the detected inhalation and exhalation periods.

21. The method of claim 18, further including:
f) detecting at least one type of abnormal user breathing based at least in part on the first sensed characteristic; and
g) adjusting the desired pressure over time to decrease the desired pressure until either a minimum pressure is reached or abnormal user breathing is detected.

22. The method of claim 18, further including:
fi sensing a second characteristic associated with the breathing gas; and
g) detecting inhalation and exhalation periods of user breathing cycles based at least in part on the second sensed characteristic;
h) adjusting the desired pressure in relation to the detected inhalation and exhalation periods;
i) detecting at least one type of abnormal user breathing based at least in part on the second sensed characteristic; and
j) adjusting the desired pressure over time to decrease the desired pressure until either a minimum pressure is reached or abnormal user breathing is detected.

23. An apparatus for providing a breathing gas to a user, including:
a headgear unit, including: i) a breathing interface, ii) an adjustable structure adapted to suitably fit the headgear unit to the user's head with the breathing interface disposed in operative relation to the user's facial area, iii) a blower motor assembly releasably attached to the adjustable structure, and iv) a plenum with a first end coupled to the breathing interface and an opposite end coupled to the blower motor assembly, the blower motor assembly, plenum, and breathing interface forming a breathing gas flow path; and
a control unit in operative communication with the headgear unit, the control unit including: i) a first sensor in operative communication with the breathing gas flow path to sense a first characteristic associated with the breathing gas, ii) a closed loop control logic in operative communication with the first sensor and the blower motor assembly to selectively control operation of the blower motor assembly based at least in part on a desired pressure for the breathing gas and the first sensed characteristic, and iii) a desired pressure logic in operative communication with the first sensor and the closed loop control logic, the desired pressure logic detecting inhalation and exhalation periods of user breathing cycles based at least in part on the first sensed characteristic, wherein the desired pressure logic is adapted to adjust the desired pressure in relation to the detected inhalation and exhalation periods;
wherein operation of the blower motor assembly provides the breathing gas to at least one user airway at an adjustable positive pressure via the breathing gas flow path.
